(19) United States
(12) Patent Application Publication
(10) Pub. No.: US 2009/0089889 A9
(43) Pub. Date: Apr. 2, 2009

(54) Neuromedin u receptor subtype 1 deficient transgenic mice and uses thereof

(76) Inventors: Howard Y. Chen, Westfield, NJ (US); Danald J. Marsh, Hillsborough, NJ (US)

Correspondence Address: MERCK AND CO., INC, P O BOX 2000, RAHWAY, NJ, 07065-0907, US

(21) Appl. No.: 11/654,155

(22) Filed: Jan. 17, 2007

Related U.S. Application Data
(60) Provisional application No. 60/760,032, filed on Jan. 18, 2006.

(51) Int. Cl. C12N 15/00; A01K 67/027; C12N 5/06

(52) U.S. Cl. 800/24; 435/354; 800/18

(57) ABSTRACT

Transgenic mice that have been engineered to be deficient in the gene encoding the neuromedin receptor subtype 1 gene. Such mice are useful in screening for receptor subtype-specific agonists and antagonists.

### FIELD OF THE INVENTION

The present invention relates to transgenic mice that have been engineered to be deficient in the gene encoding the neuromedin receptor subtype 1 gene.

### BACKGROUND OF THE INVENTION

Neuromedin U (NMU) was originally isolated from porcine spinal cord based upon its ability to contract rat uterine smooth muscle and has since been implicated in a variety of other physiological processes, including stress, nociception, inflammation, cardiovascular function and energy homeostasis.

NMU's role in the regulation of energy homeostasis is supported by both pharmacologic and genetic data. NMU inhibits food intake and increases energy expenditure when administered centrally (Howard, A D, et al., NATURE 406(6791): 70-74 (2000); Nakazato, M., et al., BIOCHEM BIOPHYS RES COMM., 277(1): 191-194 (2000); Ivanov, T R et al., ENDOCRINOLOGY, 143(10):3813-3821 (2002); and Wren, A M, et al., ENDOCRINOLOGY, 143(11): 4227-4234 (2002)). NMU-deficient mice develop obesity characterized by hyperphagia and reduced energy expenditure (Hanada, R, et al., Nature Medicine, 10(10): 1067-1073 (2004)), and transgenic mice overexpressing NMU are lean and hypophagic (Kowalski, T J, et al., J of Endocrinology, 185: 151-164 (2005)). Also, the internal energy status of an organism affects expression and release of NMU (Ivanov, 2002).

Two high affinity NMU receptors, NMUR1 and NMUR2, have been identified (Tan et al., 1998). NMUR1 is predominantly expressed in the periphery, whereas NMUR2 is primarily expressed in the brain. Recently, pharmacologic experiments have helped better define NMU's short- and long-term effects on energy homeostasis and to identify which NMU receptor(s) are involved in mediating these actions. It has been shown that acute administration of NMU either centrally or peripherally reduces food intake in mice in a dose-dependent fashion. Additionally, acute peripheral administration of NMU dose-dependently increases core body temperature in mice, suggesting that NMUR1 may also modulate energy expenditure. Chronic administration of NMU either centrally or peripherally reduces food intake, body weight and adiposity in mice, again in a dose-dependent fashion.

It has recently been discovered that the anorectic actions of centrally administered NMU are absent in NMUR2-deficient (Nmur2^{-/-}) mice, but are present in NMUR1-deficient (Nmur1^{-/-}) mice. In contrast, the anorectic actions of peripherally administered NMU are absent in Nmur1^{-/-} mice and are present in Nmur2^{-/-} mice. In Nmur1^{-/-} transgenic mice, body weight, body temperature and food intake are largely unaffected by mouse NMU-23 peptide administration. Such findings suggest that both NMUR1- and NMUR2-selective agonists, as well as neuromedin itself, may be useful for the treatment of obesity and other metabolic disorders.

Due to the recently discovered link between NMU's actions at the NMUR1, there is a need for transgenic mice engineered to be homozygous for a disruption in the NMU receptor subtype 1 gene. Such mice are of use in the study of the effect of agonists and antagonists specific to that receptor subtype, which may be useful in therapeutic applications for metabolic disorders.

### SUMMARY OF THE INVENTION

Cells and non-human transgenic mice have been engineered to be deficient in the gene encoding the Nmur1 protein.

In one embodiment of the current invention there is provided a transgenic mouse whose somatic cells and germ cells are homozygous for an altered Nmur1 gene which encodes a non-functional NMUR1 protein. Said mouse is fertile and capable of transmitting the altered Nmur1 gene to its offspring.

In another embodiment of the invention, there is provided a cell line derived from a transgenic mouse which is homozygous for an altered Nmur1 gene which encodes a non-functional NMUR1 protein.

In yet another embodiment of the present invention, there is provided a method of producing a mouse having somatic and germ cells that are either heterozygous or homozygous for an altered Nmur1 gene which encodes a non-functional NMUR1 protein, which comprises:

a) providing the altered Nmur1 gene designed to target a Nmur1 allele of mouse embryonic stem cells;

b) introducing the altered gene into mouse embryonic stem cells;

c) selecting embryonic stem cells which contain the altered gene;

d) introducing the embryonic stem cells containing the altered gene into mouse blastocysts;

e) transplanting the injected blastocysts into a pseudopregnant mouse,

f) allowing the embryo to develop to term to produce a chimeric founder transgenic mouse,

g) breeding the chimeric transgenic mouse with a wild-type mouse to obtain F1 mice heterozygous for said altered Nmur1 gene, and

h) breeding the heterozygous mice with each other to obtain mice homozygous for said altered Nmur1 gene.

In a most preferred embodiment, the introduction of step (d) is by microinjection. It is further contemplated that the transgenic cells and non-human mice of the present invention may be useful in NMUR1-based assays selecting for subtype-specific modulators of this receptor protein. Such modulators may have therapeutic applications for or be useful in the study of metabolic disorders. For example, a NMUR1 modulator may be used to treat these body weight disorders, such as a NMUR1 agonist to treat obesity or a NMUR1 antagonist to treat anorexia and related disorders.

### BRIEF DESCRIPTION OF THE DRAWING

FIG. 1 depicts a dose-dependent reduction in food intake by peripherally administered NMU, indicating that such an effect is mediated by NMUR1. Food intake (FIG. 1A) and body weight (FIG. 1B) were measured in C57BL/6 mice dosed intraperitoneally (i.p.) either with vehicle (saline) or with NMU approximately 30 minutes prior to onset of darkness. Measurements were taken about 18 hours later. * indicates p value of <0.05, n=6 per treatment group. In FIGS. 1C and 1D, food intake and body weight are measured in Nmur1 and Nmur2 mice dosed ip with either saline or 30 mg/kg (mkg) NMU about 30 minutes prior to the onset of the dark phase. Measurements were taken 18 hrs later. *, P<0.05 vs. saline, n=6 per treatment group. Nmur1 or Nmur2 mice were dosed i.p. with either vehicle (saline) or 30 mpk NMU ˜30 min. prior to the onset of the dark phase and food intake (C) and body weights (D) were measured 18 h (overnight) later. *, P<0.05 vs. corresponding vehicle; #, P<0.05 vs. Nmu1r+/+, NMU; $, P=0.052; n=10 per treatment group.

FIG. 2A describes results from an experiment where C57BL/6 mice were dosed intracerebronventricularly (i.c.v) with either vehicle (aCSF) or NMU ˜30 min. prior to the onset of the dark phase and food intake was measured 2 h later. *, P<0.05 vs. vehicle; n=12-24 per group. In FIG. 2B, Nmur1 or Nmur2 mice were dosed i.c.v. with either vehicle (aCSF) or 3 μg NMU ˜30 min. prior to the onset of the dark phase and food intake was measured 2 h later. *, P<0.05 vs. corresponding vehicle; #, P<0.05 vs. Nmur2+/+, NMU; n=14 per treatment group.

FIG. 3 describes the results from treatment of C57BL/6 mice with either vehicle (H₂O); 0.3, 1, 3 or 10 mpk NMU/day; or 10 mpk MTII/day for 7 days via Alzet micro-osmotic pumps implanted sc in the intrascapular space. 7-day cumulative effects on body weight (A), adiposity (B) and food intake (C). *, P<0.05 vs. Vehicle; n=4-10 per treatment group.

FIG. 4 depicts treatment of C57BL/6 mice with either vehicle (aCSF); 12, 36 or 120 μg NMU/day; or 4.8 μg MTII/day for 14 days via Alzet mini-osmotic pumps implanted subcutaneously in the intrascapular space and connected with a catheter to a permanent indwelling i.c.v cannula implanted in the dorsal third cerebroventricle. 14-day cumulative effects on body weight (A), adiposity (B) and food intake (C). *, P<0.05 vs. vehicle; n=5-8 per group.

FIG. 5 presents the results of an experiment where overnight fasted diet-induced obese C57BL/6 mice were dosed i.p. with either vehicle (saline); 0.3, 1, 3 or 10 mpk NMU; or 5 mpk MTII. Core temperature is plotted as change from vehicle baseline. A, hourly change. B, 6-h cumulative change.

### DETAILED DESCRIPTION OF THE INVENTION

The present invention relates to homozygous transgenic mice lacking a native neuromedin receptor subtype 1 protein (NMUR1 null; NMUR1^{-/-}). To this end, the present invention relates to animal cells which are homozygous for an NMUR1 deficiency due to a disruption in the gene(s) encoding NMUR1, as well as to transgenic mouse embryos which are NMUR1-deficient (NMUR1 null) due to a disruption in the gene(s) encoding NMUR1.

The Nmur1 knockout phenotype as developed by the present inventors displays a mild resistance to dietary-induced obesity, a small reduction in fasting-induced refeeding and a small decrease in light phase core temperature, as well as altered responses to NMU administration. This demonstrates that the NMUR1 protein is involved in the regulation of energy homeostasis. These NMUR1-deficient transgenic mice can be used to select for and test potential receptor subtype-specific modulators of NMUR1, which may be useful for methods of screening for NMUR1 modulators which affect body weight and associated methods of treating various disorders associated with inappropriate regulation of body weight.

The transgenic mice of the invention can be used in the study of the effect of modulators on the expression and activity of the Nmur1 gene and/or protein in the regulation of body weight and muscle mass as defined by lean body mass, including but not limited to disorders such as obesity, diabetes, anorexia, cachexia, syndrome X, and treatment of reduced lean body mass as it occurs in the frail elderly. Therefore, the transgenic mouse of the present invention may be utilized to determine the effect of certain modulators on the on the expression and activity of NMUR1, direct modulators of the activity of the Nmur1 gene or protein, and aspects of disorders involving regulation of body weight.

The generation of NMUR1-deficient transgenic mice aids in defining the in vivo function(s) of NMUR1, especially as related to the interaction of the NMUR1 in the regulation of body weight, as well as other indications listed herein, including but not limited to obesity (by reducing appetite, increasing metabolic rate, reducing fat intake or reducing carbohydrate craving), diabetes mellitus (by enhancing glucose tolerance, decreasing insulin resistance), hypertension, hyperlipidemia, and so forth.

An aspect of this invention is a method to obtain a mouse in which the cells lack a functional native Nmur1 gene. The method includes providing a gene for an altered form of the Nmur1 gene native to the mouse in the form of a transgene and targeting the transgene into a mouse chromosome at the place of the native Nmur1 gene or at another chromosomal location. The transgene can be introduced into the embryonic stem cells by a variety of methods known in the art, including electroporation, microinjection, and lipofection. Cells carrying the transgene can then be injected into blastocysts which are then implanted into pseudopregnant mice. In alternate embodiments, the transgene-targeted embryonic stem cells can be co-incubated with fertilized eggs or morulae followed by implantation into females. After gestation, the mice obtained are chimeric founder transgenic mice. The founder mice can be used in further embodiments to cross with wild-type mice to produce F1 mice heterozygous for the altered Nmur1 gene. In further embodiments, these heterozygous mice can be interbred to obtain the viable transgenic embryos whose somatic and germ cells are homozygous for the altered Nmur1 gene and thereby lack a functional Nmur1 gene. In other embodiments, the heterozygous mice can be used to produce cells lines.

The present invention especially relates to analysis of the complex function(s) of NMUR1 as related to obesity and diabetes by generating knock-out transgenic mice and studying how various potential modulators interact within these manipulated mice. As described herein in more detail, the native wild type gene is selectively inactivated in totipotent embryonic stem (ES) cells (such as those described herein) and used to generate the transgenic mice of the present invention. Techniques are available to inactivate or alter any genetic region to any mutation desired by using targeted homologous recombination to insert specific changes into chromosomal alleles. The present invention further relates to diploid animal cells, non-human transgenic embryos, non-human transgenic mice and non-human transgenic littermates which are heterozygous or homozygous for a disrupted Nmur1 gene resulting in deficient production of the NMUR1 protein. It is believed that this is the first report of an NMUR1 knockout mouse. The cells, embryos and non-human transgenic mice contain two chromosome alleles for NMUR1 wherein at least one of the NMUR1 alleles is mutated such that less than wild-type levels of NMUR1 activity is produced. The diploid mouse cell, embryo or non-human transgenic mice homozygous for a disrupted Nmur1 gene may show at least from about 50% to about 100% reduction in NMUR1 activity compared to a wild-type diploid cell. Alternatively, the diploid mouse cell, embryo or non-human transgenic mice heterozygous for a disrupted Nmur1 gene may show at least from about 10% to about 100% reduction in NMUR1 activity compared to a wild-type diploid cell.

The murine Nmur1 gene (see Tan, et al., 1998, incorporated herein by reference in its entirety) expresses a protein 405 amino acids in length . An Nmur1 gene that naturally occurs in the animal is referred to as the native gene, and if it is not mutant, it can also be referred to as wild-type. An altered Nmur1 gene should not fully encode the same NMUR1 as native to the host animal, and its expression product can be altered to a minor or greater degree, or absent altogether. In cases where it is useful to express a non-native Nmur1 gene in a transgenic animal in the absence of a native Nmur1 gene, it is preferred that the altered Nmur1 gene induce a null knockout phenotype in the animal. A modified Nmur1 gene with less drastic effects can also be useful and is within the scope of the present invention. The mutation may be a targeted deletion mutation, a targeted substitution mutation and/or a targeted insertion mutation. However, the preferred mutation is a deletion mutation, and especially preferred is a deletion mutation which results in a deletion of most if not all of the Nmur1 gene.

The present invention describes transgenic mice which have an altered, or preferably, completely deleted Nmur1 gene. Nmur1 gene deletions, gene modifications and or gene insertions can render the native gene nonfunctional, producing a “knockout” transgenic animal, or can lead to an NMUR1 with altered expression or activity.

The transgenic mice of the present invention can also be used as a source of cells for cell culture. These cells can be used for corresponding in vitro studies of NMUR1 expression, activity and the modulation thereof. The transgenic mice disclosed herein are useful for drug antagonist or agonist studies, for animal models of human diseases, and for testing of treatment of disorders or diseases associated with NMUR1. Transgenic mice lacking native NMUR1 are useful in characterizing the in vivo function(s) of NMUR1.

ES cells may be used as a target cell for transgene introduction. Such cells can be obtained from pre-implantation embryos cultured in vitro and fused with embryos (e.g., Evans et al., 1981, Nature 292: 154-156; Bradley et al., 1984, Nature 309: 255-258). Transgenes can be efficiently introduced into the ES cells by a variety of standard techniques such as DNA transfection, microinjection, or by retrovirus-mediated transduction. The resultant transformed ES cells can thereafter be combined with blastocysts from a non-human animal. The introduced ES cells thereafter colonize the embryo and contribute to the germ line of the resulting chimeric animal (Jaenisch, 1988, Science 240: 1468-1474). The use of gene-targeted ES cells in the generation of gene-targeted transgenic mice was described in 1987 (Thomas et al., Cell 51:503-512, (1987)) and is reviewed elsewhere (e.g., Frohman et al., Cell 56:145-147 (1989); Capecchi, Trends in Genet. 5:70-76 (1989); Wagner, EMBO J. 9:3025-3032 (1990) as well as in U.S. Pat. Nos. 5,464,764; and 5,789,215, both of which are hereby incorporated by reference. Therefore, techniques are available in the art to generate the NMUR1-deficient transgenic mice of the present invention. The methods for evaluating the targeted recombination events as well as the resulting knockout mice are also readily available and known in the art. Such methods include, but are not limited to DNA (southern) hybridization to detect the targeted allele, polymerase chain reaction (PCR), polyacrylamide gel electrophoresis (PAGE), in situ hybridization and western blots to detect DNA, RNA and protein.

It is believed that the generation of an NMUR1 knockout mouse had not been previously reported. Therefore, it was not evident that such a knockout mouse would display a distinct phenotype. As set forth infra in the Examples, the present invention demonstrates that NMUR1 knockout mice do have a phenotype which is characterized by a lack of response to the anorectic actions of NMU, and resistance to DIO and fasting-induced refeeding, indicating the involvement of this receptor in metabolic regulation.

Therefore, the present invention is shown to provide a model system consisting of transgenic mice, especially NMUR1^{-/-} mice, cells and assays that are useful in the study of aspects of the etiology of obesity as related to modulation of the NMUR1. The various assays are also useful for screening and selecting for compounds that have an effect on body weight regulation, the further study of these compounds and the possible administration of selected compounds to humans in order to regulate disorders which include but are not limited to obesity (by reducing appetite, increasing metabolic rate, reducing fat intake or reducing carbohydrate craving), diabetes mellitus (by enhancing glucose tolerance, decreasing insulin resistance), hypertension, hyperlipidemia, syndrome X and the like. While the preferred subject is a human, other mammals may be an effective host for a compound or compounds identified through the components of the present invention, including but not limited to other mammals, especially mammals of domesticated veterinary use such as canine and feline species, farm animals such as bovine, ovine, porcine, equine, caprine, rodents and additional undomesticated mammals. The finding that the NMUR1 is involved in the regulation of body fat will allow testing of selected NMUR1 agonists for direct measurements of their efficiency to modulate (decrease) body fat, thus assessing their therapeutic potential for the treatment of obesity. Most significantly, NMUR1 knockout mice can be used to test neuromedin receptor subtype-specific compounds.

The following examples are presented by the way of illustration and, because various other embodiments will be apparent to those in the art, the following is not to be construed as a limitation on the scope of the invention.

### EXAMPLE 1 GENERATION OF THE NMUR1 KNOCKOUT MOUSE

Nmur1 knockout (Nmur1^{-/-}) mice were generated using standard homologous recombination techniques. Briefly, a mouse genomic DNA library was screened with a mouse Nmur1mur cDNA probe, which was generated by PCR. One positive clone was isolated. Two regions of the clone were subcloned into pKO Scrambler NTKV-1904 (Stratagene) and a targeting vector was generated. The targeting vector was linearized by NotI restriction enzyme digestion and transformed into AB2.1 embryonic stem (ES) cells by electroporation with a Bio-Rad Gene Pulser. Transfected cells were cultured with G418 and FIAU for positive and negative selections, respectively. Approximately 500 clones were selected, and 20 correctly targeted ES cell clones were identified by Southern blot analysis. Five correctly targeted ES clones were injected into C57BL/6 blastocysts and these were implanted into pseudopregnant female mice. Several chimeric progeny gave germ-line transmission of the mutant NMUR1 allele and one NMUR1^{-/-} was established. F₃ hybrid mice were used in all experiments

### EXAMPLE 2 STRATEGY TO DELINEATE RECEPTOR SUBTYPE DIFFERENCES

Because the NMUR1 is predominantly expressed in the periphery and the NMUR2 is predominantly expressed in the central nervous system, it was necessary to develop different strategies to target receptor subtype specificity. For specific targeting of the NMUR1, the agonist was administered subcutaneously (sc) into the intrascapular space; for the NMUR2 it was administered into the cerebral ventricles (icv).

Mice were anesthetized with an intramuscular (im) injection of ketamine (100 mg/kg) and domitor (0.75 mg/kg) and placed in a stereotactic device (Kopf Instruments). For acute i.c.v. studies, a 26-gauge single acute guide cannula (Plastics One) was implanted into the dorsal third cerebral ventricle (0.22 mm posterior, 0.3 mm lateral and 3.3 mm ventral to bregma) and secured to the skull with cyanoacrylate adhesive followed by dental cement. Following surgery, a 33-gauge dummy cannula (Plastics One) was inserted into each guide cannula and mice were given an im injection of atapimazole (5 mg/kg). All cannulated mice were given one week of postoperative recovery, during which time they were handled daily to minimize nonspecific stress. All substances were administered to conscious mice with a repeating dispenser (Hamilton) equipped with a 50-μl Hamilton syringe and 33-gauge needle designed to extend 0.1-0.2 mm beyond the tip of the guide cannula. Mouse NMU-23 (Phoenix Pharmaceuticals) was dissolved in artificial cerebrospinal fluid (aCSF; Harvard Apparatus) and adjusted the pH to ˜7 with NaOH. All substances were injected in a total volume of 1 μl. Mice were given at least a 48-h recovery period between treatments. All acute icv injection studies were of crossover design, a paradigm in which mice initially treated with vehicle are subsequently treated with the agent of interest dissolved in vehicle and vice versa. For chronic icv studies, a 28-gauge osmotic pump connector cannula (Plastics One) was implanted into the dorsal third cerebral ventricle and secured to the skull. Then a micro-osmotic pump designed to deliver 0.5 μl/h (Alza Co.) and filled with aCSF was implanted subcutaneously in the intrascapular space and connected with vinyl tubing to the osmotic pump connector cannula.

Immediately following surgery, mice were given an im injection of atapimazole. Five days following the initial surgery, mice were again anesthetized and placed in the stereotactic device. Micro-osmotic pumps were removed and replaced with mini-osmotic pumps designed to deliver 0.5 μl/h (Alza Co.) and filled with either aCSF or mouse NMU-23 (Mimotopes) or MTII (Bachem) dissolved in aCSF. The pH of all solutions was adjusted to ˜7 with NaOH. Immediately following surgery, mice were given an im injection of atapimazole. For chronic sc infusions studies, mice were anesthetized and then a micro-osmotic pump designed to deliver 0.5 μl/h and filled with either sterile H₂O or mouse NMU-23 (Mimotopes) or MTII (Bachem) dissolved in sterile H₂O was implanted subcutaneously in the intrascapular space. The pH of all solutions was adjusted to ˜7 with NaOH. Immediately following surgery, mice were given an im injection of atapimazole.

### EXAMPLE 3 ASSESSMENT OF ENERGY HOMEOSTASIS

The Nmur1 mice were maintained on a 50% C57BL/6×50% 129S6/SvEv background and Nmur2 mice (licensed from Deltagen) on a 75% C57BL/6×25% 129/OlaHsd background. For comparison with wild-type, 10-12 week-old male C57BL/6 mice were purchased from Taconic Farms. Mice were individually housed in Tecniplast cages in a conventional SPF facility. Mice were maintained on either regular chow (Teklad 7012: 13.4% kcal from fat; Harlan Teklad), a moderate fat diet (D12266B: 32% kcal from fat; Research Diets, Inc.) or, to induce obesity (DIO), a high fat diet (D12492: 60% kcal from fat; Research Diets, Inc.) with ad libitum access to water in a 12-h light/12-h dark cycle, unless stated otherwise.

For acute nocturnal feeding studies, ad libitum fed male mice maintained on regular chow were weighed and dosed either intraperitoneally (ip) or icv ˜30 min prior to the onset of the dark phase of the light cycle and provided with a preweighed aliquot of chow which was then weighed two and 18 hours (overnight) after the onset of the dark phase. Mice were again weighed at the later time point. For chronic feeding studies, ad libitum fed male mice normally maintained on regular chow were switched to a palatable moderate fat diet at the initiation of treatment and food intake and body weight were measured daily.

Whole body composition analysis of conscious live mice was conducted using a Quantitative Magnetic Resonance (QMR) method (EchoMRI™, Echo Medical Systems, Houston, Tex.) (Tinsley et al. (2004) Obesity Research 12:150). The Minispec (Bruker-Optics, Billerica, Mass.) was used as NMR hardware having an applied static magnetic field corresponding to radio frequency of 7.5 MHz. Automatic tuning and calibration of the NMR instrument parameters was conducted daily for quality control.

Body temperature changes were measured as well. Male diet-induced obese mice maintained on a high fat diet were implanted with Mini-Mitter transmitters (Mini Mitter, Bend, Oreg.) prior to the study. Temperature recordings were initiated one day prior to dosing. Mice were fasted overnight and then the following morning 3 h after the onset of the light phase mice were dosed ip. Core body temperature was monitored for 6 h following dosing.

All resultant measurement values were reported as mean±S.E.M. and data was analyzed by the two-tailed unpaired Student's t test. P values≦0.05 were reported as significant.

### EXAMPLE 4 ACUTE EFFECTS OF NEUROMEDIN ON NMUR1 KNOCKOUT NMUR2 KNOCKOUT AND WILD TYPE MICE

For comparisons, Nmur2 knockout (Nmur2−/−) mice and wild type male mice were used to examine both short and long-term effects of agonist administration. As indicated supra, the wild type mice were also subjected to a DIO feeding regimen to examine agonist effects.

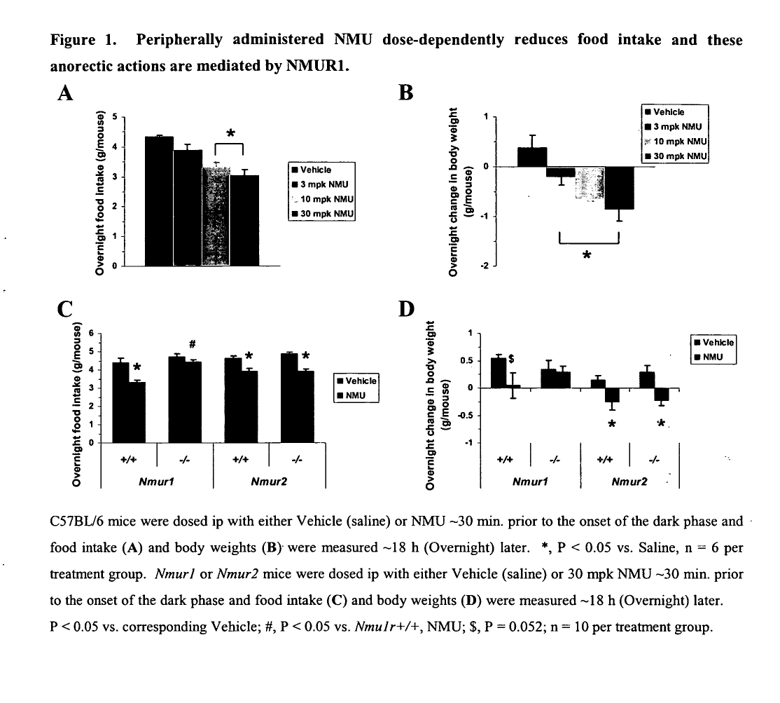

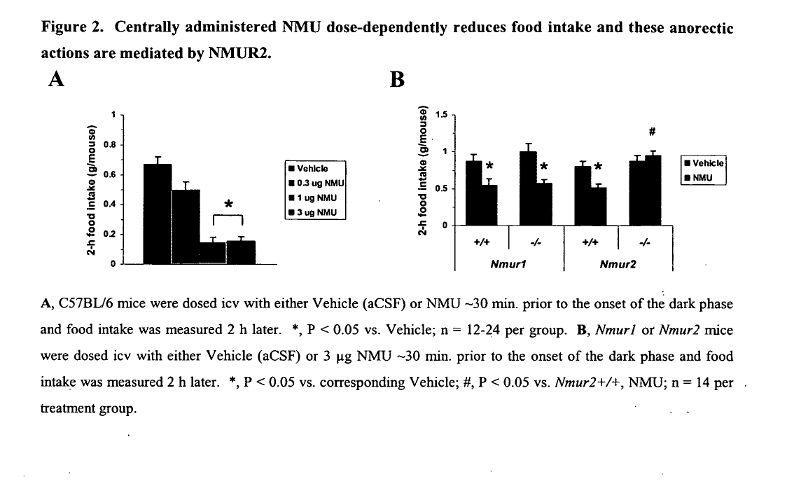

As seen in FIG. 1, there is a dose-dependent reduction in both food intake and body weight seen 18 hours following peripheral administration of NMU. This effect is not seen in NMUR1 knockout mice, which indicates that it is mediated by NMUR1. By contrast, FIG. 2 demonstrates that the food intake reduction seen with central administration of NMU is mediated via the NMUR2 receptor. Food intake is reduced in C57BL/6 mice 2 h after administration. As would be expected, Nmur2 mice do not show the same reduction (FIG. 2B).

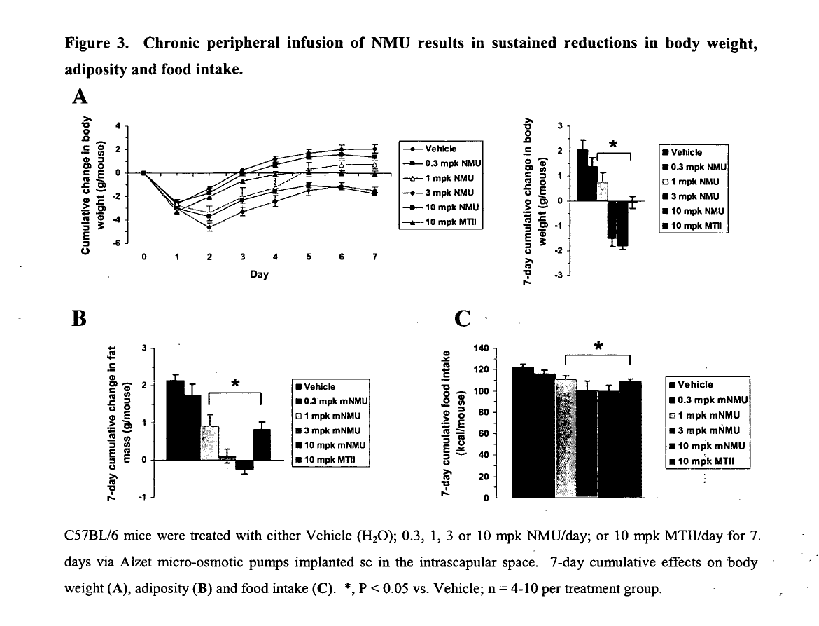

FIG. 3 describes the results from peripheral treatment of C57BL/6 mice with either vehicle (H₂O); 0.3, 1, 3 or 10 mpk NMU/day; or 10 mpk MTII/day for 7 days via Alzet micro-osmotic pumps implanted subcutaneously in the intrascapular space. 7-day cumulative, dose-dependent effects on body weight (A), adiposity (B) and food intake (C) are seen.

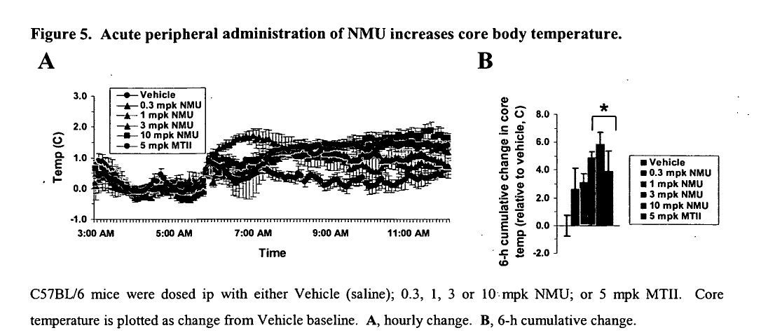

NMU administration also increases core body temperature. FIG. 5 shows changes in core temperature resulting from peripheral dosing with either saline vehicle; 0.3, 1.3, or 10 mpk NMU; or 5 mpk MTII. Core temperature is plotted as change from vehicle baseline.

In summary, the acute anorectic actions of peripherally administered NMU are mediated by NMUR1 receptors. There is also a dose-dependent effect of peripherally administered NMU on core temperature in fasted DIO mice. NMUR2 mediates the acute anorectic actions of centrally administered NMU. In addition, acute central administration of NMU also stimulates fine motor movements and grooming behavior via NMUR2 receptors (data not shown).

### EXAMPLE 5 CHRONIC EFFECTS OF NEUROMEDIN ON NMUR1 KNOCKOUT, NMUR2 KNOCKOUT AND WILD TYPE MICE

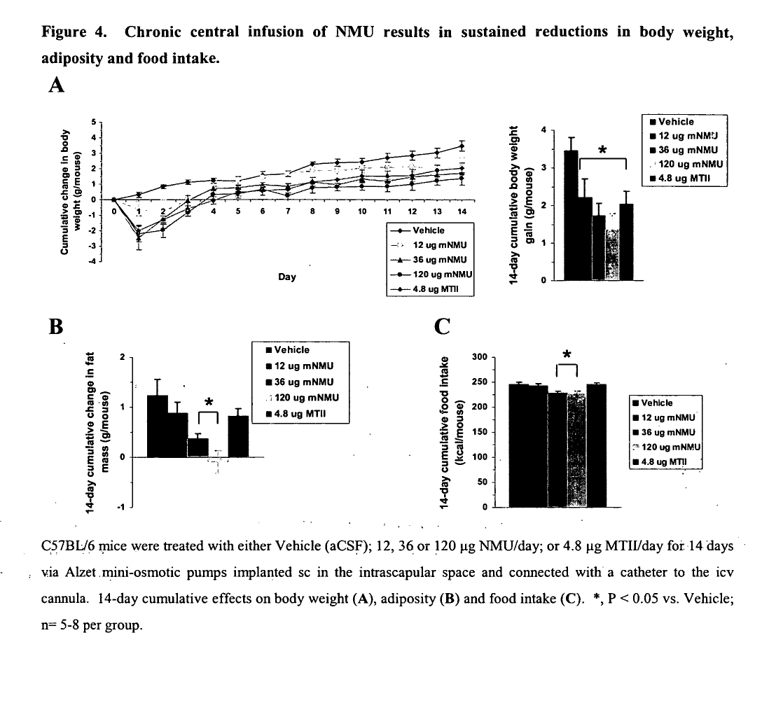

As seen in FIG. 4, effects of NMU persist over 14 days of central administration. C57BL/6 mice were treated with either aCSF vehicle; 12, 36 or 120 μg NMU/day; or 4.8 μg MTII/day and dose-dependent cumulative effects on body weight (A), adiposity (B) and food intake (C) are seen. Effects on food intake and body weight are also seen following 7-day chronic s.c. infusion of mouse NMU-23 in C57BL/6 mice. Effects are also seen following 14-day chronic i.c.v. infusion of mouse NMU-23 on body weight; body composition; food intake; and on motor activity. Additionally, 13-day chronic subcutaneous infusion of NMU has demonstrable effects on body weight; food intake and core temperature in DIO mice. The effects of a rat NMU peptide (rNMU-23) on ad libitum food intake of lean and DIO C57BL/6 mice are seen to mimic those of the mouse isoform.

Other embodiments are fully within the scope of the following claims. All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. All such variations apparent to those skilled in the art are deemed to be within the spirit, scope and concept of the invention as defined by the appended claims.

1. A transgenic mouse whose somatic cells and germ cells are homozygous for a disrupted or deleted native Nmurl gene, which renders the native Nmurl gene non-functional, and wherein the transgenic mouse displays a phenotype selected from the group consisting of resistance to dietary-induced obesity, reduction in fasting-induced refeeding, and decrease in light phase core temperature.

2. The mouse of claim 1, which is capable of reproducing.

3. A cell line derived from the transgenic mouse of claim 1.

4. A method of producing a mouse having somatic and germ cells that are homozygous for a disrupted or deleted native Nmurl gene wherein the disruption or deletion renders the native Nmurl gene non-functional and the mouse displays a phenotype selected from the group consisting of resistance to dietary-induced obesity, reduction in fasting-induced refeeding, and decrease in light phase core temperature, which comprises:
(a) providing a vector designed to target a NMUR1 allele of mouse embryonic stem cells and disrupt or delete the native Nmurl gene at the NMUR1 allele;
(b) introducing the vector into mouse embryonic stem cells to disrupt or delete the Nmurl gene in the NMUR1 allele of the embryonic stem cells;
(c) selecting embryonic stem cells which contain the the disrupted or deleted native Nmurl gene in the NMUR1 allele;
(d) introducing the embryonic stem cells containing the the disrupted or deleted native Nmurl gene into mouse blastocysts;
(e) transplanting the injected blastocysts into a pseudopregnant mouse,
(f) allowing the embryo to develop to term to produce a chimeric founder transgenic mouse,
(g) breeding the chimeric transgenic mouse with a wild-type mouse to obtain F1 mice heterozygous for said disrupted or deleted native Nmurl gene, and
(h) breeding the heterozygous mice with each other to obtain mice homozygous for said disrupted or deleted native Nmurl gene.

5. The method of claim 4 wherein the introduction of step (d) is by microinjection.
